(19) United States
(12) Patent Application Publication
(10) Pub. No.: US 2018/0071141 A1
(43) Pub. Date: Mar. 15, 2018

(54) INTRAOCULAR OXYGEN DELIVERY AND ABSORPTION DEVICES AND METHODS

(71) Applicant: The Regents of the University of Colorado, a body Corporate, Denver, CO (US)

(72) Inventor: Jeffrey Olson, Denver, CO (US)

(73) Assignee: The Regents of the University of Colorado, a body Corporate, Denver, CO (US)

(21) Appl. No.: 15/564,664

(22) PCT Filed: Apr. 6, 2016
(86) PCT No.: PCT/US2016/026138

§ 371 Date: Oct. 5, 2017

Related U.S. Application Data
(60) Provisional application No. 62/143,537, filed on Apr. 6, 2015.

(51) Int. Cl. A61F 9/00; A61K 9/00; A61K 33/00

(52) U.S. Cl. CPC A61F 9/0017; A61K 9/0051; A61F 2250/0067; A61F 2230/0069; A61K 33/00

(57) ABSTRACT

Example embodiments comprise an implantable ophthalmic device comprising at least one shell encapsulating high-oxygen affinity substance and designed to increase the oxygen partial pressure in an oxygen-deprived structure of, or space within, an eye. Example embodiments may be used therapeutically to treat ischemic conditions in situ and thereby prevent damage, for example, retinal damage.

### BACKGROUND

Certain ocular conditions are characterized as being ischemic, or having low oxygen states, such as diabetic retinopathy and retinal vein and artery occlusion, whereas others are characterized by oxidative damage from high oxygen levels, such as age-related macular degeneration, cataract, and glaucoma. The ability to modulate the oxygen tension within a vitreous cavity would ameliorate the effect of these pathological conditions.

What is therefore needed is a novel intraocular implant that would make possible the reversal of ischemic conditions and the associated vision loss. The present disclosure addresses this need.

### SUMMARY

Example embodiments comprise an implantable ophthalmic device designed to increase the oxygen partial pressure (i.e., oxygen tension or concentration) in an oxygen-deprived structure of, or space within, an eye. An example implantable ophthalmic device comprises at least one shell encapsulating high-oxygen affinity substance. Example methods are also disclosed and may comprise implanting one or a plurality of example implantable ophthalmic devices. Example embodiments may be used therapeutically to treat ischemic conditions in situ and thereby prevent damage, for example, retinal damage. Further, in disease states where oxygen levels are too high, such as macular degeneration or cataract, an implantable ophthalmic device can shunt oxygen away from tissues and into an oxygen sink such as the choriocapillaris or suprachoroidal space.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure.

FIG. 1 illustrates an implantable ophthalmic device in accordance with an example embodiment;

FIG. 2 illustrates another implantable ophthalmic device in accordance with an example embodiment;

FIG. 3 illustrates example implantable ophthalmic devices implanted in an eye.

### DETAILED DESCRIPTION

Persons skilled in the art will readily appreciate that various aspects of the present disclosure may be realized by any number of methods and apparatuses configured to perform the intended functions. Stated differently, other methods and apparatuses may be incorporated herein to perform the intended functions. It should also be noted that the accompanying drawing figures referred to herein are not all drawn to scale, but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

While example embodiments will be described with regard to ischemic conditions of the eye, example embodiments may apply more generally to other ischemic conditions and/or chronic shortages of oxygen, whether or not in the eye, for example, in connection with skin, organ or other tissue grafts where increased oxygenation is needed.

Example embodiments comprise an implantable ophthalmic device designed to increase the oxygen partial pressure in a vitreous cavity of an eye by delivering oxygen from an area of higher oxygen partial pressure to an area of lower oxygen partial pressure. The delivery can be by diffusion. In various embodiments, the delivery is passive, not active. As a result, example embodiments may enhance oxygen delivery to an oxygen-deprived structure of, or space within, an eye, for example, a retina. Example embodiments may be used therapeutically to treat ischemic conditions in situ and thereby prevent damage, for example, retinal damage. Further, in disease states where oxygen levels are too high, such as macular degeneration or cataract, an implantable ophthalmic device can shunt oxygen away from tissues and into an oxygen sink such as the choriocapillaris or suprachoroidal space.

An example implantable ophthalmic device does not comprise a biological component for the production of oxygen. An example implantable ophthalmic device does not comprise an electrical component for the production of oxygen. For example, an example implantable ophthalmic device does not incorporate the use of electrolysis as a method for oxygenation; rather, it is passive. Notwithstanding the foregoing, an example implantable ophthalmic device may comprise electrical or biological components to modulate the flow of the high-oxygen affinity substance through the device, or to modulate the amount of oxygen absorbed or transferred.

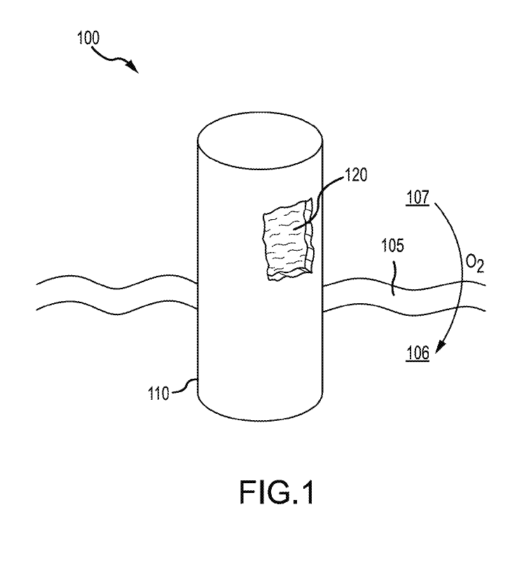

With reference to FIG. 1, an example implantable ophthalmic device 100 comprises at least one shell 110, and a high-oxygen affinity substance 120. In one example embodiment, shell 110 encapsulates high-oxygen affinity substance 120. In various example embodiments, implantable ophthalmic device 100 is configured to provide additional oxygen to an oxygen-deprived structure of, or space within, an eye.

Shell 110 is generally any biocompatible structure configured to provide a preferred pathway or otherwise allow the passage of gases (e.g., oxygen and carbon dioxide exchange), but also prevent the high-oxygen affinity substance from migrating outward. Stated another way, shell 110 may comprise any structure configured to confine high-oxygen affinity substance while allowing oxygen to be delivered therethrough. The shell may have any shape, be any suitable size, and comprise any biocompatible material.

Example shapes of shell 110 include spherical, cylindrical tubular, elongate tubular, a capsule, a coil, cubical, conical, frustoconical, toroidal, etc. By way of example, a loop can be formed by shell 110, providing a continuous pathway for diffusion of oxygen from an area of higher oxygen partial pressure to an area of lower oxygen partial pressure.

In other embodiments, implantable ophthalmic device 100 can comprise a reservoir, bulb or the like which may be compressible on either end, allowing for a greater surface area for gas exchange.

In yet other example embodiments, any component that is conventionally implanted in the eye can further incorporate all or an individual component of implantable ophthalmic device 100. Thus, for example, an intraocular lens can comprise shell 110. In other embodiments, glaucoma drainage devices, drug delivery systems, hardware for retinal re-attachment, corneal transplants, intracorneal hardware, and/or implants placed in eyelids for cosmetic or exposure reasons can comprise shell 110.

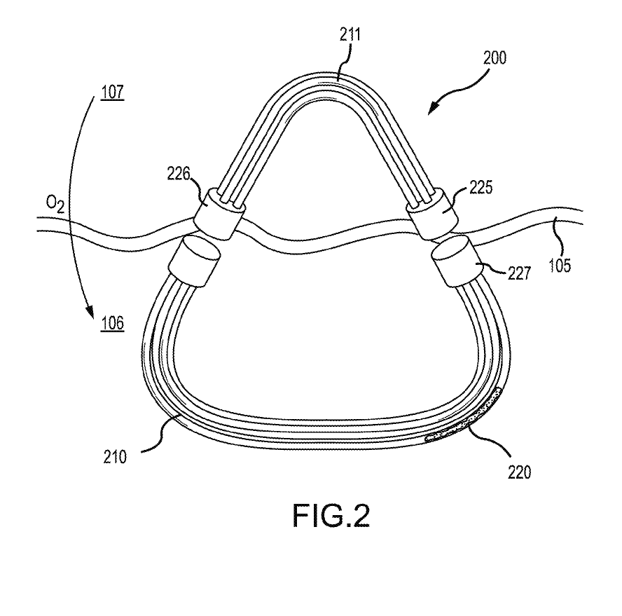

By way of example, and with momentary reference to FIG. 2, an example implantable ophthalmic device 200 comprises a plurality of shells 210, and a high-oxygen affinity substance 220. Plurality of shells 210 can be arranged in any shape that is elliptical (e.g., circles, ovals, ellipses, and the like), non-elliptical (e.g., triangles, rectangles, squares, hexagons, trapezoids, pentagons, stars, and the like), or random.

Shell 110, in various embodiments, can comprise one or more walls or conduits within the lumen of shell 110 to add a delay to the exchange of oxygen, and thereby provide for a more consistent exchange of oxygen over an extended period of time.

In example embodiments, a shape having a smaller crossing profile is selected. In example embodiments, it may be desirable to maximize surface area for gas exchange. In other example embodiments, the shape of a shell of an implantable ophthalmic device intended for longer use may have a smaller surface-area-to-volume ratio to encapsulate more high-oxygen affinity substance in a smaller space. In still other example embodiments, the shape of a shell of an implantable ophthalmic device intended for shorter use may have a larger surface-area-to-volume ratio to expose more high-oxygen affinity substance to an oxygen-deprived structure or space.

The size of shell 110 may be selected to encapsulate a desired amount of high-oxygen affinity substance. In various embodiments, the lumen of the shell has a volume of from about 50 μL to about 500 μL, more preferably from about 100 μL to about 300 μL, most preferably about 250 μL. In various embodiments, the lumen of the shell has an internal diameter of from about 0.1 mm to about 4 mm, more preferably from about 0.2 mm to about 1 mm, or about 0.75 mm. However, in other embodiments, e.g., embodiments comprising a coil or bundle of elongate tubular shells as discussed infra, each individual shell can have a diameter of about 10 μm to about 100 μm. In various embodiments, the shell has a length of from about 1 mm to about 10 mm, more preferably from about 1 mm to about 7 mm, or about 5 mm. The size and length may be selected so as to not impinge the vision of a patient when implanted in a vitreous cavity of his/her eye.

Generally speaking, shell 110 may comprise any biocompatible, immunologically inert material(s). More specifically, the shell may comprise a rigid material, such as plastics, metals, alloys and the like, or a flexible material, such as silicones, nitriles, nylons, polycarbonates, polyethylenes, polypropylenes and the like. An example shell may comprise a plurality of materials.

In example embodiments, all or a portion of shell 110 can comprise a material that can be punctured to adjust the concentration and/or volume of high-oxygen affinity substance. For example, some or all of high-oxygen affinity substance could be replaced with fresh high-oxygen affinity substance or diluted with a solvent. In this regard, the material can be configured to self-seal upon removal of the puncturing tool.

Further, an example high-oxygen affinity substance can be pre-oxygenated to very high levels before placing into an example implantable ophthalmic device. Additionally, an example high-oxygen affinity substance can be periodically exchanged or re-exposed to high oxygen levels to refill its oxygen carrying capacity. This can be done either on a portion of the implantable ophthalmic device exposed to the vitreous, a portion crossing the cornea or sclera, or a portion within the vitreous cavity. This can be accomplished by injecting fresh high-oxygen affinity substance directly into the implantable ophthalmic device, injecting oxygen into the implantable ophthalmic device, or exposing a membrane of the example implantable ophthalmic device to oxygen which can then diffuse into the high-oxygen affinity substance.

In other example embodiments, shell 110 can comprise a porous membrane or other material that is porous. In example embodiments, a portion of the shell (e.g., a central portion, one or more bands about the shell, one or more “windows” through the shell, etc.) can comprise a porous membrane or other material that is porous. As used herein, the term “porous” generally refers to being permeable to exchange of gases (e.g., oxygen and carbon dioxide). By way of non-limiting example, a porous membrane may be configured to allow the passage of gases (e.g., oxygen and carbon dioxide exchange), but also prevent the high-oxygen affinity substance from migrating outward.

Example porous materials for use in connection herewith may comprise various filtration membranes known in the art, including NF-90 nanopore membranes and biologically inert reconstituted cellulose membranes (e.g., microdialysis fibers and hollow bore membranes). Still additional porous materials can comprise one or more of poly (vinylidene fluoride) (PVDF), nylon, regenerated cellulose, dialysis membranes, osmosis/reverse osmosis membranes, biotissues (e.g., amniotic membrane, lens capsule, Bruch's membrane, etc.), and the like. Example porous materials may have an average pore size of from about 1 kDa to about 50 kDa, more preferably from about 10 kDa to about 20 kDa, most preferably from about 13 kDa to about 18 kDa.

In example embodiments, shell 110 comprises an anchor (e.g., a suture clip or the like comprising any suitable material) designed to secure the implantable ophthalmic device during and/or after implantation. In example embodiments, the anchor secures the implantable ophthalmic device to an adjacent tissue or structure (e.g., via a suture). In yet other example embodiments, the anchor secures a plurality of example implantable ophthalmic devices together.

In example embodiments, and with momentary reference to FIG. 2, an example implantable ophthalmic device 200 can comprise a coupling 225. Coupling 225 can be located anywhere along or at an end of a shell, or secure a plurality of shells 211 together. In one example embodiment, plurality of shells 211 are arranged in parallel with each other (e.g., a plurality of HF fibers, or other coil or bundle of elongate tubular shells). In this example, the ends of plurality of shells 211 can each be connected to coupling 225. Moreover, plurality of shells 211 can each be connected to coupling 225 on one end and another coupling 226 on the other end. Moreover, in another example embodiment, coupling 225 can be aligned or otherwise adjacent to or in proximity with at least one other coupling 227. In this embodiment, coupling 227 is not connected to plurality of shells 211. Instead, coupling 227 is connected to plurality of shells 210. Thus, coupling 225 can comprise a porous material, such as those described supra, and can be generally configured to facilitate the delivery of oxygen from one shell or plurality of shells to another. In other embodiments, a coupling is not required to facilitate the delivery of oxygen from one shell or plurality of shells to another. Rather, the mere proximity is sufficient to accomplish the same.

Thus, in an example embodiment, an implantable ophthalmic device can comprise two or more separate shells or pluralities of shells, wherein each shell or plurality of shells is connected to another via couplings associated with each shell or plurality of shells. In the illustrated embodiment of FIG. 2, an example first plurality of shells is connected to an example second plurality of shells via respective couplings at the ends of the first and second pluralities of shells. In this regard, a loop can be formed, providing a continuous pathway for diffusion of oxygen from an area of higher oxygen partial pressure to an area of lower oxygen partial pressure.

With continued reference to FIG. 1, in example embodiments, shell 110 is all or partially coated with a therapeutic agent, such as a drug, antiproliferative, antithrombotic, etc. Such embodiments may be especially advantageous when an example implantable ophthalmic device is not implanted in a vitreous cavity of an eye but at or near another oxygen-deprived structure of, or space within, an eye, such as an anterior chamber or under conjunctiva, to name just a few.

With reference again to FIG. 1, high-oxygen affinity substance 120 generally includes any and all high-oxygen affinity substances, for example, perfluroctane and other fluorocarbon based materials, hemoglobin and hemoglobin derivatives, hyperbranched polymer-protected porphryins, stem cells, dendrimers, micelles, placental umbilical cord blood, hemerythrin, respirocytes, and other similar substance such as those used as blood-replacement fluids. In general, any high-oxygen affinity substance can be used in accordance with the present disclosure.

In some embodiments, an otherwise “inert” high-oxygen affinity substance can be externally activated, e.g., by light, temperature, the passage of time, ultrasound, electric current, or exposure of the implantable ophthalmic device itself to high oxygen, such as a small cap that could be placed over the scleral end of the implantable ophthalmic device to “recharge” the material and increase its oxygen content, to name a few.

An example concentration or amount of high-oxygen affinity substance 120 is generally that sufficient to exchange enough local oxygen to increase the oxygen partial pressure at or near an oxygen-deprived structure of, or space within, an eye, by at least 1 mmHg, 2 mmHg, 4 mmHg or more, preferably to an otherwise “normal” range. A normal range, in accordance with various embodiments, may be at least about 10 mmHg, more preferably at least about 15 mmHg, most preferably about 20 mmHg, or an otherwise normal or improved oxygen partial pressure. Notwithstanding the foregoing, an example concentration or amount of high-oxygen affinity substance 120 is generally that sufficient to exchange enough local oxygen to increase the oxygen partial pressure at or near an oxygen-deprived structure of, or space within, an eye, could be as high as 100 mmHg in certain clinical situations.

As referenced above, in accordance with various embodiments of the present disclosure, a first portion of an implantable ophthalmic device shell is configured to be implanted at a first location within an eye (e.g., exterior to the sclera and under the conjunctiva) having a first oxygen partial pressure, and a second portion of the implantable ophthalmic device shell is configured to be implanted at a second location within the eye (e.g., a vitreous cavity) having a second oxygen partial pressure that is measurably lower than the first oxygen partial pressure. In such embodiments, oxygen is exchanged from the first portion to the second portion by diffusion to measurably increase the second oxygen partial pressure.

With reference again to FIG. 1, implantable ophthalmic device 100 can be configured to deliver oxygen from an area of higher oxygen partial pressure 107 to an area of lower oxygen partial pressure 106. Likewise, and with reference again to FIG. 2, implantable ophthalmic device 200 can be configured to deliver oxygen from an area of higher oxygen partial pressure 107 to an area of lower oxygen partial pressure 106.

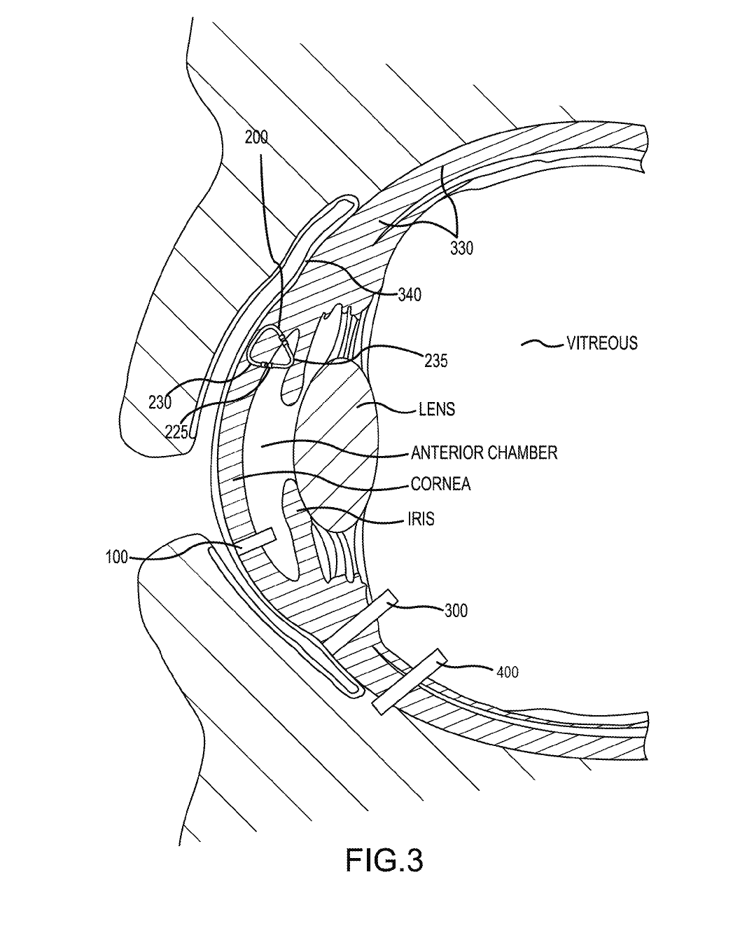

With reference now to FIG. 3, area of higher oxygen partial pressure 107 can be, among other locations, exterior to the sclera 330, under the conjunctiva 340. Area of lower oxygen partial pressure 106 can be, among other locations, the anterior chamber, the vitreous or other vitreous cavity. In various embodiments, an example, implantable ophthalmic device is positioned in the eye so as to not indirectly or directly in the visual axis, or otherwise impinge the vision of a patient. For example, an example device may be implanted in the peripheral cornea near the liubbus, or even more peripheral, going across the sclera.

In an example embodiment, a portion of implantable ophthalmic device 100 is implanted beneath the conjunctiva 340 and external to the sclera 330, and the high-oxygen affinity substance acts as a counter-current exchange mechanism, taking up oxygen beneath the conjunctiva 340, and delivering oxygen within the eye. This diffusion of oxygen, through the high-oxygen affinity substance, can be modulated or even interrupted by restricting the flow inside the lumen by use of valves, channels, or shunts, that augment or diminish the flow of the high-oxygen affinity substance through implantable ophthalmic device 100. In other embodiments, the permeability of the shell membrane to oxygen and/or other gases can be controllably modulated via light, thermal, or other mechanisms. Further, the shell membrane can have an additional outer shell or sheath, which may be mechanically retractable in order to create this augmenting effect. For example, the exposure of the membrane, at a location external to the sclera, can be changed. In another example embodiment, the exposure of the membrane, at a location internal to the sclera, can be changed.

In some embodiments, area of higher oxygen partial pressure 107 is all or partially separated by barrier 105 from area of lower oxygen partial pressure 106. Barrier 105 can be a tissue, for example, the eye wall or sclera. In such embodiments, implantable ophthalmic device 100 can all or partially straddle barrier 105. That is, implantable ophthalmic device 100 can extend from one side to another side of barrier 105 and beyond. In some embodiments, one or more couplings located on a first side of barrier 105 can be aligned or otherwise adjacent to or in proximity with at least one or more couplings located on a second side of barrier 105. In this regard, one portion of implantable ophthalmic device 100 can be on one side of a barrier 105 and another portion of implantable ophthalmic device 100 can be on another side of a barrier 105.

In various embodiments, the exchange oxygen and carbon dioxide is unidirectional, while in other embodiments, the exchange is bidirectional.

With reference to FIG. 3, implantable ophthalmic device 100 can be placed via the pars plana, with a portion implanted exterior to the sclera 330, under the conjunctiva 340. Another portion can extend into the anterior chamber. In this regard, oxygen can diffuse into implantable ophthalmic device 100 from an area of higher oxygen partial pressure to the anterior chamber.

Similarly, implantable ophthalmic device 300 can be placed via the pars plana, with a portion implanted exterior to the sclera 330, under the conjunctiva 340. Another portion can extend into the vitreous. In this regard, oxygen can diffuse into implantable ophthalmic device 300 from an area of higher oxygen partial pressure to the vitreous.

In another embodiment, implantable ophthalmic device 400 can be implanted with a portion extending across the sclera 330, near the conjunctiva 340. Another portion can extend into the vitreous. As above, oxygen can diffuse into implantable ophthalmic device 400 from an area of higher oxygen partial pressure to the vitreous.

In yet another embodiment, implantable ophthalmic device 200 can be placed via the pars plana, with a first portion 230 implanted exterior to the sclera 330, under the conjunctiva 340. A second portion 235 can extend into the anterior chamber. First portion 230 and second portion 235 can each have at least one porous coupling 225, each of which is aligned or otherwise adjacent to or in proximity with at least one other porous coupling 225 at or near a barrier. In this regard, oxygen can diffuse into implantable ophthalmic device 200 from an area of higher oxygen partial pressure to an anterior chamber through the plurality of porous couplings 225 of implantable ophthalmic device 200.

Thus, in example embodiments, an implantable ophthalmic device comprises a shell for implanting within an eye, the shell comprising a first portion and a second portion, and a high-oxygen affinity substance encapsulated within the shell. In such embodiments, the first portion and the second portion each comprise a porous membrane to allow the passage of oxygen and to prevent the passage of the high-oxygen affinity substance. Moreover, the high-oxygen affinity substance is in contact with the porous membrane of each of the first portion and the second portion to exchange oxygen by diffusion from a first location (having a first oxygen partial pressure) to a second location (having a second oxygen partial pressure that is measurably lower than the first oxygen partial pressure) to measurably increase the second oxygen partial pressure. In such embodiments, the first location can be exterior to the shell and adjacent to the porous membrane of the first portion, and the second location can be exterior to the shell and adjacent to the porous membrane of the second portion.

Example methods comprise delivering and implanting one or a plurality of example implantable ophthalmic devices. In various embodiments, an example implantable ophthalmic device is implanted in the anterior chamber or other vitreous cavity, external to the sclera, or a combination thereof. In some embodiments, at least a portion of an example implantable ophthalmic device is implanted within a vitreous cavity, while at least another portion extends out of such vitreous cavity to an area of higher oxygen partial pressure. Put another way, in various embodiments, the entirety of an example implantable ophthalmic device is not in contact with vitreous fluid. In general however, an example implantable ophthalmic device may be implanted at or near any oxygen-deprived structure of, or space within, an eye, such as an anterior chamber or under conjunctiva, or in the wall of a membrane, to name just a few.

Surgical access to an oxygen-deprived structure or space may be accomplished according to methods known in the art, for example, through a pars plana incision.

Example methods also comprise anchoring an example implantable ophthalmic device at or near an oxygen-deprived structure of, or space within, an eye. The step of anchoring may comprise suturing, clipping, tying or the like. In example embodiments, the step of anchoring may comprise anchoring an example implantable ophthalmic device in the periphery of a vitreous cavity of a patient's eye so as to not impinge her/his vision. The anchoring may be permanent or temporary.

Example methods further comprise increasing the oxygen partial pressure at or near an oxygen-deprived structure of, or space within, an eye by at least about 1%, more preferably at least about 5%, most preferably at least about 10%. Example methods further comprise increasing the oxygen partial pressure at or near an oxygen-deprived structure of, or space within, an eye by at least 1 mmHg, 2 mmHg, 4 mmHg or more, to at least about 10 mmHg, more preferably at least about 15 mmHg, most preferably about 20 mmHg, or an otherwise normal or improved oxygen partial pressure. Example methods still further comprise increasing the oxygen partial pressure at or near an oxygen-deprived structure of, or space within, an eye for at least about 2 weeks, more preferably at least about 3-6 months, most preferably at least about 1 year or more. Moreover, an example implant can be implanted in the an permanently, and the amount of oxygen delivered held constant, modulated manually, or adjusted automatically depending upon the clinical need.

A prototype of an example implantable ophthalmic device similar to that illustrated in FIG. 2 was constructed. In a first test, water was injected through a plurality of HF fibers, and after 120 minutes exhibited 40% loss attributed to evaporation. In a second test, by comparison, perfluroctane was injected through the plurality of HF fibers, and after 120 minutes exhibited only 3% loss, which was not attributed to evaporation.

To further test the device described, six devices composed of cellulose microdialysis fiber bundle membranes were used. Three active devices were filled with perfluroctane which was measured to have an average partial pressure of 186 mmHg. Three control devices were filled with normal saline which had been processed through a membrane contractor to remove oxygen. The average partial pressure of oxygen for the saline implants was 52 mmHg.

Each device was placed into a closed container of saline which had a baseline partial pressure of oxygen of 52 mmHg. Each device was placed through an opening such that half the device was exposed to room air with a partial pressure of oxygen of 184 mmHg, while the other half remained in solution. All six devices were left in place for 90 minutes, after which the partial pressure of oxygen in the saline container was measured.

In the active group, the final average partial pressure of oxygen of the saline in the container was 92 mmHg, a statistically significant increase of 40 mmHg of oxygen (p-value<0.001), demonstrating that the perfluroctane acted as a carrier, delivering oxygen across a gradient. In the control group, the final average partial pressure of the saline in the container was 54 mmHg, a non-statistically significant change of 2 mmHg of oxygen from baseline (p-value=0.5).

Additional in vitro data was collected for example implantable ophthalmic devices. Eight 4 ml closed containers were filled with saline solution, and 0.5 mg of sodium sulfite mixed in to deoxygenate the solution. The starting oxygen concentration in each was <2 mmHg as measured by non-consumptive, fiber optic instrumentation. The perfluroctane (PFO) filled microdialysis fibers of the oxygenation device of the present disclosure were then placed into four of the containers. Pure oxygen was bubbled through the solution of PFO just prior to testing in order to raise the oxygen level over 200 mmHg. The remaining four containers were the control group, with each having empty microdialysis fibers placed inside.

With reference to Table 1 below, with the oxygenation device of the present disclosure, there was a pronounced increase in the saline oxygen levels over eight hours, with a final average value of 60.2 mmHg. There was a slight increase in the oxygen level of the control group, from the small amount of room air inside the container which was exposed to the saline, with a final average value of 12.6 mmHg. This difference was statistically significant (p<0.05).

TABLE 1

| | 5 | 15 | 30 | 45 | 2 | 5 | 8 |
|---|---|---|---|---|---|---|---|
| | min | min | min | min | hours | hours | hours |
| Oxygen | 1.67 | 1.98 | 2.29 | 7.1 | 20.3 | 48.5 | 60.2 |
| Concentration | | | | | | | |
| in Solution | | | | | | | |
| (mmHg) | | | | | | | |
| Oxygenation | | | | | | | |
| device | | | | | | | |
| Oxygen | 1.94 | 0.59 | 6.03 | 6.6 | 7 | 7.8 | 12.6 |
| Concentration | | | | | | | |
| in Solution | | | | | | | |
| (mmHg) | | | | | | | |
| Control | | | | | | | |

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

I claim:

1. An implantable ophthalmic device comprising:
a shell comprising a porous membrane, and
a high-oxygen affinity substance encapsulated within the shell,
wherein a first portion of an implantable ophthalmic device is configured to be implanted at a first location within an eye having a first oxygen partial pressure, and a second portion of the implantable ophthalmic device is configured to be implanted at a second location within the eye having a second oxygen partial pressure that is measurably lower than the first oxygen partial pressure, and
wherein oxygen is exchanged from the first portion to the second portion by diffusion to measurably increase the second oxygen partial pressure.

2. The implantable ophthalmic device of claim 1, wherein the first location is exterior to a sclera and under a conjunctiva of the eye and the second location is one of an anterior chamber and a vitreous.

3. The implantable ophthalmic device of claim 1, wherein the shell has one of the following shapes: spherical, cylindrical tubular, elongate tubular shape, or capsule.

4. The implantable ophthalmic device of claim 1, wherein the porous membrane comprises biologically inert reconstituted cellulose.

5. The implantable ophthalmic device of claim 1, wherein the porous membrane has an average pore size of about 13 kDa.

6. The implantable ophthalmic device of claim 1, wherein the porous membrane is configured to allow oxygen and carbon dioxide exchange.

7. The implantable ophthalmic device of claim 1, wherein the porous membrane is configured to prevent the high-oxygen affinity substance from migrating out of the implantable ophthalmic device.

8. The implantable ophthalmic device of claim 1, wherein the high-oxygen affinity substance is perfluroctane.

9. A method for therapeutically treating ischemic conditions in situ comprising the steps of:
delivering a first portion of an implantable ophthalmic device to a location that is exterior to a sclera and under a conjunctiva of an eye, and a second portion of the implantable ophthalmic device to a vitreous cavity of the eye, wherein the implantable ophthalmic device comprises:
a shell comprising a porous membrane;
a high-oxygen affinity substance encapsulated within the shell.

10. The method of claim 9 further comprising delivering the implantable ophthalmic device through a pars plana incision.

11. The method of claim 9 further comprising increasing the oxygen partial pressure within the vitreous cavity by at least about 10%.

12. The method of claim 9 further comprising increasing the oxygen partial pressure within the vitreous cavity to about 20 mmHg.

13. The method of claim 9 further comprising increasing the oxygen partial pressure within the vitreous cavity for at least about 1 year or more.

14. The method of claim 9, wherein the shell has one of the following shapes:
spherical, cylindrical tubular, elongate tubular shape, or capsule.

15. The method of claim 9, wherein the porous membrane comprises biologically inert reconstituted cellulose, wherein the porous membrane has an average pore size of about 13 kDa, wherein the porous membrane is configured to allow oxygen and carbon dioxide exchange, and wherein the porous membrane is configured to prevent the high-oxygen affinity substance from migrating out of the implantable ophthalmic device.

16. The method of claim 9, wherein the high-oxygen affinity substance is perfluroctane.

17. An implantable ophthalmic device comprising:
a shell for implanting within an eye, the shell comprising a first portion and a second portion; and
a high-oxygen affinity substance encapsulated within the shell;
wherein the first portion and the second portion each comprise a porous membrane to allow the passage of oxygen and to prevent the passage of the high-oxygen affinity substance;
wherein the high-oxygen affinity substance is in contact with the porous membrane of each of the first portion and the second portion to exchange oxygen by diffusion from a first location having a first oxygen partial pressure to a second location having a second oxygen partial pressure that is measurably lower than the first oxygen partial pressure to measurably increase the second oxygen partial pressure;
wherein the first location is exterior to the shell and adjacent to the porous membrane of the first portion; and
wherein the second location is exterior to the shell and adjacent to the porous membrane of the second portion.
